(19) United States
(12) Patent Application Publication
(10) Pub. No.: US 2021/0031182 A1
(43) Pub. Date: Feb. 4, 2021

(54) KIT HAVING PERIPHERAL-CIRCLE COMPATIBLE SCALING STRUCTURE FOR TESTING SOMATIC CELLS IN RAW MILK

(71) Applicant: KIKOH CORPORATION, Mie (JP)

(72) Inventors: Toru SEKIGUCHI, Mie (JP); Aiko WATANABE, Mie (JP); Eriko WATANABE, Mie (JP)

(21) Appl. No.: 16/966,461

(22) PCT Filed: Jan. 29, 2019
(86) PCT No.: PCT/JP2019/002957

§ 371 Date: Jul. 30, 2020

(30) Foreign Application Priority Data
Jan. 31, 2018 (JP) 2018-015498

(51) Int. Cl. B01L 3/00; C12Q 1/44; G01N 33/04

(52) U.S. Cl. CPC B01L 3/5023; B01L 2300/0681; G01N 33/04; C12Q 1/44

(57) ABSTRACT

The present invention provides a laminated filter, a sample testing tool, and a kit for detecting and/or quantifying a test target substance in a sample (in particular, a liquid sample, for example, raw milk) in a simple manner and with sufficiently high measurement sensitivity and measurement precision. The present invention relates to a laminated filter for detecting and/or quantifying a test target substance in a sample (in particular, a liquid sample, for example, raw milk), wherein the laminated filter contains a plurality of filters, and at least two of the plurality of filters contain a reaction reagent that reacts with the test target substance.

### TECHNICAL FIELD

The present invention relates to a laminated filter, a sample testing apparatus, and a kit for detecting and/or quantifying a substance subjected to testing in a sample (especially in a liquid sample). Specifically, the present invention relates to a laminated filter, a sample testing apparatus, and a kit for testing somatic cells (e.g., leucocytes in raw milk (e.g., raw cow milk).

### BACKGROUND ART

The number of dairy farming households in Japan is 18600 households, and 892800 dairy cattle are being raised. Although the total number of dairy farming households is in decline, the number of cattle per household is increasing. While dairy farmers are constantly striving to stabilize the business by producing high quality raw milk, the dairy farmers have no other choice but to confront mastitis, which is a “disease” that diminishes the quality of raw milk. Contraction of mastitis by dairy cattle typically results in the following problems: (1) somatic cells (especially leukocytes) increase in the raw milk and cause a change in milk components to diminish the quality of the raw milk; (2) mastitis reduces the quantity of raw milk production, i.e., milk output of cows decreases, leading to loss in quantity of milk; and (3) since raw milk containing a large number of somatic cells leads to diminished productivity and quality of dairy products, the price at which milk is purchased by Japan Agricultural Cooperatives or dairy product manufacturers is lowered, which becomes the loss of the farmers. If the somatic cell count in 1 milliliter of raw milk exceeds 200,000, the milk price is lowered by 5 yen to up to 40 yen per kilogram. The current milk price is 78 to 80 yen per kg.

90% of the revenue of dairy farmers is from sale of raw milk. Income generated per dairy cow is on average about 167000 yen. Once mastitis is contracted, loss per cow is reportedly 87000 yen, which would be a loss of half of the revenue. Currently, economic loss due to mastitis is reportedly 30 billion yen just in Hokkaido, and 2 billion dollars annually in the US. Therefore, early discovery and treatment of mastitis is a pressing issue for dairy farmers. It is not an exaggeration to say that resolution of mastitis would lead to stability in business.

Meanwhile, highly precise measurement of the somatic cell count in raw milk requires an expensive apparatus known as a flow cytometer (8 to 20 million yen), which makes ownership difficult for a dairy farmer. Further, test kits that can be used by a dairy farmer onsite have low sensitivity and precision, such that it would be challenging to determine contraction of mastitis.

85% to 90% of somatic cells in raw milk is leukocytes. Once mastitis is contracted, such somatic cells in raw milk increases, and the quality of the raw milk deteriorates. A hydrolase known as esterase in leukocytes can be used for conveniently measuring somatic cells in raw milk or the like. A test kit for estimating the somatic cell count in raw milk by measuring the enzymatic activity of such esterase has been conceived (Patent Literature 1: Japanese National Phase PCT Laid-open Publication No. 2005-526513). The feature of Patent Literature 1 is in using a filter medium and a film with a three layer structure and a design placing a perforated film on the top portion, applying and immobilizing a substrate that reacts with leukocyte esterase on the next film, capturing somatic cells in raw milk, and providing a place for reacting with the substrate. Filter paper for absorbing raw milk that is not needed for the reaction, components that inhibit reactions of esterase contained in the raw milk, or the like is also disposed. During an actual measurement of somatic cells, raw milk is pipetted, a certain amount thereof is dropped into a hole on the top portion, somatic cells are filtered by a filter on the second layer and captured, and unneeded raw milk is allowed to be absorbed by the filter paper on the third layer. Furthermore, a buffer referred to as an activator is dropped in from the hole on the top portion, and the condition is changed to the optimal reaction condition for esterase of leukocytes, and the measurer waits for an enzymatic reaction to start. Eventually, the enzyme substrate applied to the filter is degraded by leukocyte esterase. The color of the filter changes due to a degradation product. The change in color is determined through visual inspection or a specific equipment to estimate the number of somatic cells contained in raw milk. The somatic cell count aids in the diagnosis of mastitis.

While Patent Literature 1 is relatively convenient, the sensitivity and precision of measurement cannot be considered sufficient. In view of such a backdrop, there is a demand for the development of a laminated filter, a sample testing apparatus, and a kit for testing that readily measures the somatic cell count in raw milk onsite with a high level of sensitivity and precision.

An immunochromatographic test chip that is widely used in the diagnosis of influenza or the like is a diagnostic chip prepared to detect and then diagnose a virus by applying an antibody that reacts with an influenza virus on what is known as a membrane filter of nitrocellulose or the like applied on a support and allowing a bodily fluid comprising an influenza virus to flow therethrough. Meanwhile, a reaction takes place on a single sheet and at a single location. If a contained component is to be detected at a lower concentration by increasing the sensitivity of detection of a target component with these chips, it was necessary to concentrate a corresponding component from a test material. There was a limit to increasing only the detection sensitivity of a diagnostic chip.

### CITATION LIST

### Patent Literature

[PTL 1] Japanese National Phase PCT Laid-open Publication No. 2005-526513

### SUMMARY OF INVENTION

### Technical Problem

The technical problem to be solved of the present invention is to provide a laminated filter, a sample testing apparatus, and a kit for detecting and/or quantifying a substance subjected to testing in a sample (especially in a liquid sample) with a sufficient level of measurement sensitivity and precision as well as convenience. For example, the technical problem to be solved of the present invention is to provide a laminated filter, a sample testing apparatus, and a kit for testing somatic cells in raw milk.

### Solution to Problem

The problems described above were solved by the following:

### (Item 1)

A laminated filter for detecting and/or quantifying a substance subjected to testing, the laminated filter comprising a plurality of filters, wherein at least two of the plurality of filters comprise a reaction reagent that reacts with the substance subjected to testing.

### (Item 2)

The laminated filter of item 1, wherein the plurality of filters comprise filters with different shapes.

### (Item 3)

The laminated filter of item 1 or 2, wherein the plurality of filters are at least four filters.

### (Item 4)

The laminated filter of any one of items 1 to 3, wherein the plurality of filters comprise a first filter and a second filter, the first filter being n-gonal, and the second filter being m-gonal or circular, wherein n is an integer from 3 to 6 and m is greater than n.

### (Item 5)

The laminated filter of item 4, wherein the first filter is adjacent to the second filter.

### (Item 6)

The laminated filter of item 4 or 5, wherein the first filter and the second filter have different areas.

### (Item 7)

The laminated filter of any one of items 4 to 6, wherein the laminated filter comprises a plurality of the first filters and a plurality of the second filters, and at least one of the first filters is adjacent to at least one of the second filters.

### (Item 8)

The laminated filter of item 5 or 7, wherein the first filter and the second filter are alternately laminated.

### (Item 9)

The laminated filter of item 7 or 8, wherein at least two of the plurality of the first filters are laminated after being rotated so that positions of vertices are different.

### (Item 10)

The laminated filter of any one of items 7 to 9, wherein the plurality of the second filters are m-gonal, and at least two of the second filters are laminated after being rotated so that positions of vertices are different.

### (Item 11)

The laminated filter of any one of items 4 to 9, wherein the first filter is quadrangular, and the second filter is circular.

### (Item 12)

The laminated filter of any one of items 1 to 11, wherein the filter is a filter selected from the group consisting of filter paper, non-woven fabric, and glass fiber.

### (Item 13)

The laminated filter of any one of items 1 to 12, wherein a reaction between the substance subjected to testing and the reaction reagent is a color reaction.

### (Item 14)

The laminated filter of any one of items 1 to 13, wherein the substance subjected to testing is an enzyme, and the reaction reagent comprises a substrate of the enzyme.

### (Item 15)

The laminated filter of any one of items 1 to 14, wherein the substance subjected to testing is esterase, and the reaction reagent comprises a substrate of esterase.

### (Item 16)

The laminated filter of item 15, wherein the reaction reagent comprises 3-[N-(p-toluenesulfonyl)-L-alanyloxy]indole.

### (Item 17)

The laminated filter of any one of items 1 to 16, wherein at least one of the plurality of filters comprises a reaction promoting agent.

### (Item 18)

A sample testing apparatus comprising the laminated filter of any one of items 1 to 17 and a housing for the laminated filter.

### (Item 19)

The sample testing apparatus of item 18 for measuring a leukocyte in raw milk.

### (Item 20)

The sample testing apparatus of item 18 or 19, wherein the housing comprises a syringe.

### (Item 21)

A kit comprising the sample testing apparatus of any one of items 18 to 20.

The present invention relates to a laminated filter, a sample testing apparatus, and a kit for not only testing a somatic cell in raw milk, but also detecting and/or quantifying a substance subjected to testing in a sample (especially in a liquid sample) with a high level of sensitivity/precision as well as convenience.

### Advantageous Effects of Invention

The present invention provides a laminated filter, a sample testing apparatus, and a kit for detecting and/or quantifying a substance subjected to testing in a sample (especially in a liquid sample) with a sufficient level of measurement sensitivity and precision as well as convenience. For example, the present invention provides a laminated filter, a sample testing apparatus, and a kit for testing a somatic cell in raw milk. The present invention provides a laminated filter, a sample testing apparatus, and a kit for testing a somatic cell in raw milk with a sufficient level of measurement sensitivity and precision as well as convenience in accordance with the present invention.

### BRIEF DESCRIPTION OF DRAWINGS

FIG. 1 shows an example of a housing for housing the laminated filter of the invention (closed without comprising a laminated filter).

FIG. 2 shows an example of a housing for housing the laminated filter of the invention (open without comprising a laminated filter).

FIG. 3 shows an example of a housing for housing the laminated filter of the invention (closed while comprising a laminated filter).

FIG. 4 shows an example of a housing for housing the laminated filter of the invention having a circular filter laminated on a quadrangular filter (open while comprising a laminated filter).

FIG. 5 shows an example of lamination of circular filters and quadrangular filters in the preparation of the laminated filter of the invention.

FIG. 6 shows an exemplary color chart used in measurement that uses the laminated filter of the invention and a calibration sample. The color shade of each square is associated with a range of cell concentrations in a calibration sample (or diluted calibration sample).

### DESCRIPTION OF EMBODIMENTS

The embodiments of the invention are described hereinafter. It should be understood that the embodiments are exemplification of the invention, so that the scope of the invention is not limited to such preferred embodiments. It should be understood that those skilled in the art can readily make a modification or change within the scope of the invention by referring to the following preferred Examples.

(1. Laminated Filter)

The laminated filter of the invention for detecting and/or quantifying a substance subjected to testing comprises a plurality of filters, and at least two of the plurality of filters comprise a reaction reagent that reacts with the substance subjected to testing. A laminated filter preferably comprises 4 or more, 5 or more, 6 or more, or 7 or more filters, and more preferably comprises 4 or more filters.

As a filter, any filter that provides a place for a substance subjected to testing to react with a reaction reagent and enables the substance subjected to testing to move from a filter to an adjacent filter when laminated can be used. Examples of filters that can be used include, but are not limited to, filter paper, non-woven fabric, and glass fiber. Examples of materials with which non-woven fabric can be made include, but are not limited to, polypropylene, polyester, nylon, and combinations thereof.

A filter contained in the laminated filter of the invention is a filter through which a liquid test sample passes or is filtered. The pore size of a filter used in the present invention is 0.1 micrometer to 100 micrometer, preferably 0.2 micrometer to 10 micrometer, more preferably 0.2 micrometer to 5 micrometer, and most preferably 0.2 micrometer to 1.0 micrometer. The diameter of a fiber constituting a filter used in the present invention is 0.05 micrometer to 300 micrometer, preferably 0.1 micrometer to 50 micrometer, more preferably 0.1 micrometer to 30 micrometer, and most preferably 0.1 micrometer to 10 micrometer.

A plurality of filters contained in a laminated filter are preferably filters with different shapes. For example, a plurality of filters comprise a first filter and a second filter. The first filter is n-gonal, and the second filter is m-gonal or circular, preferably circular, wherein n is an integer from 3 to 6 (preferably 4 or 5, and most preferably 4), and m is greater than n. A filter does not necessarily need to have rotational symmetry. For example, if a first filter is quadrangular, the first filter does not necessarily need to be square. If a second filter is circular, the second filter does not necessarily need to be a true circle. In addition to the first filter with a first shape and the second filter with a second shape, the laminated filter of the invention can further comprise a third and/or fourth filter with yet another shape.

A first filter and a second filter of a laminated filter may or may not be adjacent. If a laminated filter comprises a plurality of first filters and a plurality of second filters, at least one of the first filters may or may not be adjacent to at least one of the second filters. Preferably, first filters and second filters are alternately laminated. When laminated alternately, another filter may be interposed between a first filter and a second filter.

A first filter and a second filter of a laminated filter do not necessarily need to have the same area. It is rather preferable to have different areas (ratio has a variable magnification). For example, if the first filter is quadrangular and the second filter is circular, the circular filter can have a size where it is nearly inscribed in the quadrangular filter, or a size where it is nearly circumscribed.

If a laminated filter comprises a plurality of first filters, at least two of the first filters are laminated after being rotated so that positions of vertices are different. For example, if a laminated filter comprises a first filter A and a first filter B, the filters can be laminated after rotating the vertices of filter A to positions that are about 30 degrees or about 45 degrees different from the vertices of filter B. Alternatively, if a laminated filter comprises a plurality of first filters, at least two of the first filters can be laminated so that positions of vertices overlap.

If a laminated filter comprises a plurality of second filters, at least two of the second filters can be laminated after being rotated so that positions of vertices are different, or laminated so that positions of vertices overlap.

The sensitivity/precision of measurement can be adjusted in the laminated filter of the invention by appropriately adjusting the number of filters laminated, the shape(s) of filters used, material of filters, concentration of reaction reagent added on filters, whether a reaction promoting agent is added on filters (amount and concentration thereof when added), order of laminating filters, orientation upon laminating filters, or the like.

Examples of laminated filters used in the present invention include, but are not limited to, laminated filters with a peripheral-circle variable magnification structure. Specifically, the laminated filter used in the present invention can be prepared by laminating a plurality of types of filters with different shapes, such as a side of a quadrangular filter and a circular filter. The areas of a plurality of types of filters with different shapes used to prepare the laminated filter of the invention do not necessarily need to have equal magnification, but can have irregular variable magnifications. For example, the laminated filter of the invention can be prepared by laminating a side of a quadrangular filter and a circular filter with different areas (see FIGS. 4 and 5).

(2. Additional Filter for Sample Filtration)

Any liquid sample can be used as a sample that can be used in the laminated filter of the invention. Examples thereof include, but are not limited to, biological materials (e.g., blood, urine, saliva, spinal fluid, semen, bodily fluid, and breast milk). When a sample requires filtration such as when measuring esterase in raw milk, any filter with an effect of filtering out clumps or cells contained in a sample such as raw milk can be added to the front surface or top portion of the laminated filter. For example, the laminated filter of the invention can comprise a first additional filter for removing clumps that are larger than cells and/or a second additional filter for removing cells.

A first additional filter has, for example, a mesh size which allows cells (e.g., leukocytes) to pass through but filters out clumps that are larger than cells (e.g., leukocytes). Examples of mesh sizes of a first additional filter include, but are not limited to, 200 and 300 mesh. A first additional filter typically allows 40 micrometer or smaller particles, 35 micrometer or smaller particles, 30 micrometer or smaller particles, 25 micrometer or smaller particles, 20 micrometer or smaller particles, 15 micrometer or smaller particles, or 10 micrometer or smaller particles to pass through but not particles with a greater size. Representative examples of materials of a first additional filter include, but are not limited to, non-woven fabric, nylon mesh, and polycarbonate.

A second additional filter has a mesh size that allows clumps smaller than cells (e.g., leukocytes) to pass through, but not cells (e.g., leukocytes). Examples of mesh sizes of a second additional filter include, but are not limited to, 3μ, 5μ, and 10μ. A second additional filter typically allows 15 micrometer or smaller, 12 micrometer or smaller, 10 micrometer or smaller, 8 micrometer or smaller, 6 micrometer or smaller, 5 micrometer or smaller, 4 micrometer or smaller, 3 micrometer or smaller, 2 micrometer or smaller, 1 micrometer or smaller, and 0.5 micrometer or smaller particles to pass through but not particles with a greater size. The material of a second additional filter can be the same or different from the material of a first additional filter. Representative examples of materials of a second additional filter include, but are not limited to, non-woven fabric, rayon filter, polycarbonate, and paper. A second additional filter may or may not have a positive charge on the surface thereof to capture leukocytes. Examples of functional groups imparting such a charge include, but are not limited to, primary amine, secondary amine, tertiary amine, cationic surfactant, and corona treatment.

(3. Substance Subjected to Testing and Reaction Reagent)

A substance subjected to testing for the laminated filter of the invention is not limited. Any substance that can react on a filter contained in a laminated filter can be used.

Examples of the substances subjected to testing of the invention include, but are not limited to, enzymes, substrates of an enzymatic reaction, antibodies, antigens, and dyes. Examples of antigens include, but are not limited to, viruses, bacteria, cells, cell fragments, cell components, DNAs, RNAs, proteins, sugar chains, and lipids. If, for example, the substance subjected to testing of the invention is an enzyme, a reaction reagent comprises a substrate of an enzymatic reaction. If, for example, the substance subjected to testing of the invention is a substrate of an enzymatic reaction, a reaction reagent comprises an enzyme. If, for example, the substance subjected to testing of the invention is an antibody, a reaction reagent comprises an antigen. If, for example, the substance subjected to testing of the invention is an antigen, a reaction reagent comprises an antibody.

If, for example, a substance subjected to testing is esterase in a sample, any substance that exhibits color by an enzymatic reaction of esterase (esterolysis reaction or proteolysis reaction) can be used as the esterase substrate. Examples of substrates of esterase include, but are not limited to, 3-[N-(p-toluenesulfonyl)-L-alanyloxy]indole, 3-[N-(toluene-4′-sulfonyl)-L-alanyloxy]-indole, 3-[N-(diphenylcarbamoyl)-L-alanyloxy]-indole, 3-[N-acetyl-L-alanyloxy]indole, 3-[N-formyl-L-alanyloxy]-indole, 3-[N-(benzyloxycarbonyl)-L-alanyloxy]-indole, 3-[N-(benzoyl)-D,L-alanyloxy]-indole, 3-[N-(5′,5′-dimethyl-3′-oxo-cyclohexen-1′-yl)-L-alanyloxy]-indole, 3-[N-(2′-nitrobenzene-sulfenyl)-L-alanyloxy]-indole, 3-[N-(p-toluenesulfonyl)-L-alanyloxy]-5-phenylpyrrole, 3-(N-acetyl-L-alanyloxy)-5-phenylpyrrole, 1-[N-(p-toluenesulfonyl)-L-alanyloxy]naphthalene, 3-[N-(p-toluenesulfonyl)-L-alanyloxy]-1-methoxynaphthalene, thiazole-2-azo-4′-[1′-(N-benzyloxycarbonyl-L-alanyloxy)-5′-methoxynaphthalene], thiazole-2-azo-1′-[2′-(N-benzyloxycarbonyl-L-alanyloxy)naphthalene], thiazole-2-azo-4′[1′-(N-benzyloxycarbonyl-L-alanyloxy)-naphthalene], 2-methoxy-4-nitro-benzene-azo-4′-[1′-(N-benzyloxycarbonyl-L-alanyloxy)naphthalene], 6-methoxy-benzothiazole-2-azo-2′-[1′(N-benzyloxycarbonyl-L-alanyloxy)-naphthalene], 2,5-dimethoxy-benzene-azo-4′-[1′-(N-benzyloxycarbonyl-L-alanyloxy)-naphthalene], 2,4-dinitro-benzene-azo-4′-[1′-(N-benzyloxycarbonyl-L-alanyloxy)-benzene], 1-[N-(p-toluenesulfonyl)-L-alanyloxy]-3-methoxybenzene, di-acetyl-3′,3′-dibromo-5′,5′-dichloro-phenolsulfonephthalein, di-acetyl-3′,5′,3′,5′-tetrabromo-phenylsulfonephthalein, di-acetyl-4,5,6,7,3′,5′,3′,5′-octabromo-phenolsulfonephthalein, di-(N-benzylcarbonyl-L-alanyl)-3′,5′,3′,5′-tetrabromo-phenolsulfonephthalein, di-(N-benzyloxycarbonyl-L-phenylalanyl)-3′,5′,3′,5′-tetrabromo-phenolsulfonephthalein, and 3-[N-(p-tosylalanyloxy)-4-nitrobenzene.

(4. Reaction Promoting Agent)

The filter of the invention can optionally comprise a reaction promoting agent. If, for example, a substance subjected to testing or reaction reagent comprises esterase, an esterase enzymatic reaction is promoted by a reaction promoting agent, which is alcohol represented by R—OH wherein R is substituted with at least one halogen atom. Preferably, the number of carbons of R is 6 or greater. Examples of reaction promoting agents include, but are not limited to, 6-chloro-1-hexanol, 6-bromo-1-hexanol, 2-chlorocyclohexanol, 2-bromocyclohexanol, 4-bromocyclohexanol, 1-chloro-2-heptanol, 7-chloro-1-heptanol, 8-bromo-1-octanol, 11-bromo-1-undecanol, 12-bromo-1-dodecanol, and 2-chlorocyclohexanol.

(5. Housing)

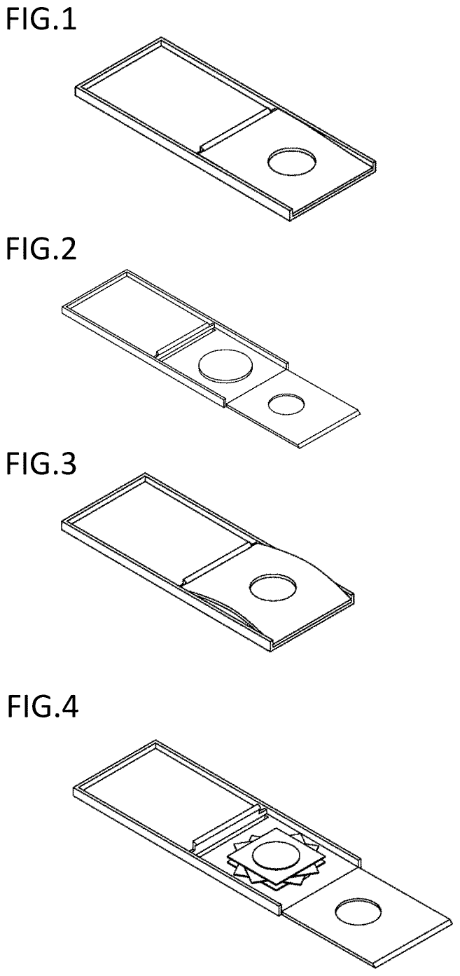

Preferably, the laminated filter of the invention can comprise the plurality of filters described above within a housing. A part of a housing can be film-like (e.g., cover film). A housing preferably has a window for observing a color reaction and a protrusion for pressing on the entire non-woven fabric to keep the thickness constant and to keep the amount of sample absorbed constant. The window for observing a color reaction can also function as an inlet for a sample subjected to measurement. The laminated filter of the invention is preferably stored in a sealed film (e.g., aluminized film). The laminated filter of the invention is preferably stored in an vacuum state. A housing can be, for example, the housing shown in FIG. 1, a part of a syringe, or a cartridge coupled to a syringe.

(6. Addition of an Enzymatic Reaction Substrate to a Filter)

If a substance subjected to testing is an enzyme (e.g., esterase in a sample), a reaction reagent (e.g., enzymatic reaction substrate) can be typically added to the filter of the invention by immersing the filter in a reaction reagent solution (e.g., solution of an enzymatic reaction substrate). Alternatively, a solution of an enzymatic reaction substrate can be added to the filter. Examples of concentrations of a reaction reagent solution (e.g., solution of an enzymatic reaction substrate) used for immobilizing a substrate to a filter include, but are not limited to, 0.08 mg/ml, 0.10 mg/ml, 0.11 mg/ml, 0.12 mg/ml, 0.13 mg/ml, 0.14 mg/ml, 0.15 mg/ml, 0.16 mg/ml, 0.17 mg/ml, 0.18 mg/ml, 0.19 mg/ml, 0.20 mg/ml, 0.3 mg/ml, 0.4 mg/ml, 0.5 mg/ml, 0.6 mg/ml, 0.7 mg/ml, 0.8 mg/ml, 0.9 mg/ml, and 1.0 mg/ml.

(7. Addition of a Reaction Promoting Agent to a Filter)

Examples of concentrations of a reaction promoting agent used when adding the reaction promoting agent to the filter of the invention include, but are not limited to, 1.0-fold, 1.2-fold, 1.4-fold, 1.6-fold, 1.8-fold, 2.0-fold, 2.2-fold, 2.4-fold, 2.6-fold, 2.8-fold, 3.0-fold, 3.5-fold, 4.0-fold, 4.5-fold, 5.0-fold, 6.0-fold, 7.0-fold, and 8.0-fold of the concentration of a reaction reagent solution (e.g., solution of an enzymatic reaction substrate).

(8. Use of a Laminated Filter)

The method of using the laminated filter of the invention is not particularly limited, but a substance subjected to testing can be detected and/or quantified simply by, for example, adding a sample of a liquid to one of the surfaces of the laminated filter (the additional filter side when comprising an additional filter for filtering a sample). When the laminated filter of the invention is housed within a housing, the entire plurality of filters is pressed down by a protrusion of the housing, which keeps the thickness of the filter constant. For this reason, the amount of liquid sample absorbed into the laminated filter remains constant. Thus, when a certain amount or more of a liquid sample is added to the laminated filter of the invention, only a certain amount of the liquid sample is absorbed into the laminated filter. As a result, cells can be detected/measured with a high level of sensitivity and precision.

Alternatively, the method of using the laminated filter of the invention is not particularly limited, but a substance subjected to testing can be detected/measured simply by, for example, immersing the filter in a liquid sample (e.g., raw milk) and retrieving the filter. When the laminated filter of the invention is housed within a housing, the entire plurality of filters is pressed down by a protrusion of the housing, which keeps the thickness of non-woven fabric constant. For this reason, the amount of raw milk absorbed into the laminated filter remains constant. Thus, the laminated filter of the invention can detect/measure cells with a high level of sensitivity and precision simply by immersing the filter in a liquid sample (e.g., raw milk) and retrieving the filter.

The laminated filter of the invention can also be disposed within a syringe or within a cartridge coupled to a syringe.

When utilizing a color reaction, a substance subjected to testing in a sample can be detected and/or quantified by visually inspecting a laminated filter directly from outside. Alternatively, coloring can be measured by well-known means. A color developed by a reaction on a plurality of filters, when viewed through a window for reaction on a housing, would be amplified as a clear color, which can be measured with a higher level of sensitivity and precision.

Since a certain period of time is required for a liquid of a sample to move from a filter to an adjacent filter, the difference in the degree of reaction (e.g., color reaction) on a plurality of filters contained in a laminated filter can reflect chronological changes in the reaction. For example, if reactions in a plurality of filters contained in a laminated filter are compared after a certain period of time has passed since adding a sample to the laminated filter, a filter close to the side where the sample was added is a reflection of a result of a reaction of a longer period of time, and a filter located away from the side where the sample was added is a reflection of a result of a reaction of a shorter period of time. Therefore, the laminated filter of the invention can also detect chronological changes of a reaction by measuring a single point over time.

(9. Measurement of Somatic Cell Count in Raw Milk)

The somatic cell count in raw milk (e.g., raw cow milk) can be measured or tested conveniently by using the laminated filter of the invention. The following is a representative measurement method.

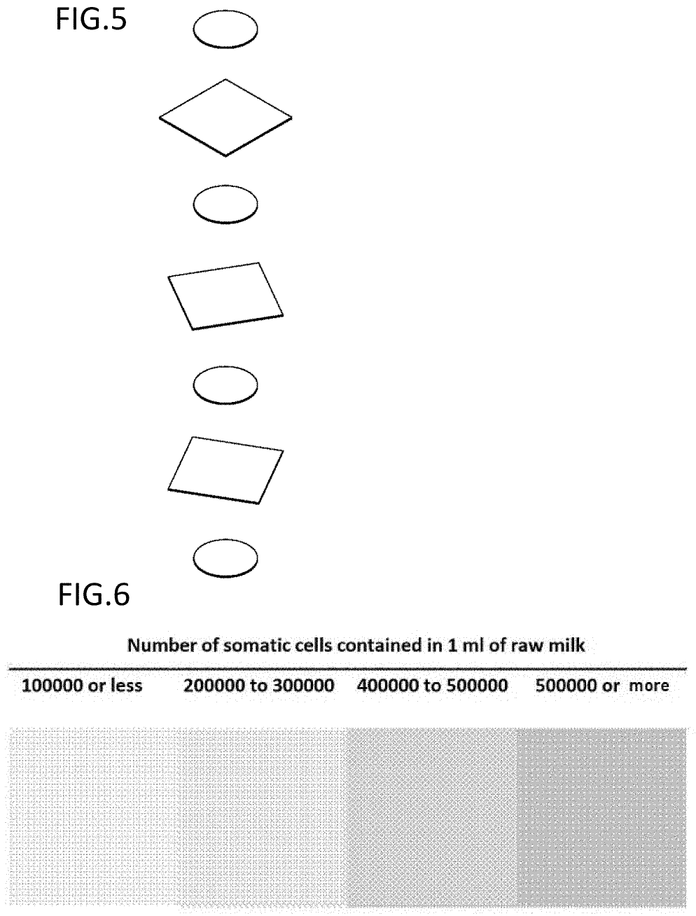

(1) A calibration sample is prepared. A calibration sample can be prepared, for example, by measuring a given sample using a standard method characterized by measuring the leukocyte count in raw milk with a flow cytometer. Examples of flow cytometers that can be used include an apparatus named FOSSMATIC manufactured by FOSS (Denmark). Typically, a sample used as a calibration sample is raw milk whose contained somatic cell count has been measured. The counted somatic cells are typically leukocytes. A plurality of calibration samples including cells of various concentrations are prepared based on a calibration sample whose cell count has been determined.
(2) A color chart showing levels of color tones is prepared and used for comparison with a color reaction due to the laminated filter of the invention. The color chart described above is an indicator showing the level of color shade corresponding to the color developed by the color reaction. For example, a color chart showing four levels of color shades is prepared (see the squares with four different shades in FIG. 6).
(3) The calibration samples comprising cells of various concentrations prepared in (1) are subjected to the laminated filter of the invention to induce a color reaction due to an enzymatic reaction of esterase. The results of the color reaction are compared with the color chart of (2) to determine the cell count corresponding to each color shade in the color chart. For example, the cell count corresponding to the lightest color (e.g., leftmost in FIG. 6), the cell count corresponding to the next darker color (e.g., 2^{nd} from the left in FIG. 6), the cell count corresponding to the next darker color (e.g., 3^{rd} from the left in FIG. 6), and the cell count corresponding to the darkest color (e.g., rightmost in FIG. 6) are determined and associated with each color shade in the color chart. In this manner, a color chart associating color shades with cell counts can be prepared as shown in FIG. 6.
(4) A sample subjected to measurement is subjected to the laminated filter of the invention to induce a color reaction. A sample can be suitably diluted with a cell-free solution as needed. It should be noted that in such a case, the reaction conditions (e.g., (i) whether the laminated filter of the invention is immersed in a sample, or a certain amount of sample is added, (ii) reaction time, (iii) reaction temperature, (iv) room temperature incubation period (e.g., period of enzymatic reaction) after completion of the reaction (e.g., after completion of step (i)), (v) temperature of sample used (e.g., room temperature, animal body temperature, refrigeration temperature, or the like), (vi) time from sample collection to measurement, and the like) should be substantially the same.
(5) The color reaction induced as a result of (4) is observed and associated with one of the shades in the color chart of (3).
(6) The cell count corresponding to the color shade associated in (5) is determined to be the cell count in the sample subjected to measurement.

### EXAMPLES

The present invention is described in detail with the following Examples and the like, but the present invention is not limited thereto.

### Example 1: Laminated Filter for Efficiently Detecting Somatic Cells in Raw Milk

Non-woven fabric, prepared from polypropylene, with a pore size of 0.2 micrometer to 10 micrometer and a fiber diameter of 0.1 micrometer to 10 micrometer was used as a filter. 0.015 ml of solution comprising a reaction reagent 3-[N-(p-toluensulfonyl)-L-alanyloxy]indole (hereinafter, referred to as TAL) (35 mg/ml), which is an esterase enzymatic reaction substrate, and a reaction promoting agent 12-bromo-1-dodecanol (hereinafter, referred to as DODECA) (50 mg/ml) was added to the filter (quadrangle with a 12 mm side), and the filter was air-dried. On both sides of the filter produced in this manner, a filter free of a reaction reagent and reaction promoting agent was laminated to prepare a laminated filter. Furthermore, to be compatible with this laminated filter, two quadrangular filters impregnated with 1.0 M Tris-HCl buffer were laminated so that corners thereof would not overlap, and a circular filter impregnated with the same buffer was further laminated thereunder. The laminated filter was then housed within the housing shown in FIGS. 1 to 2. FIG. 3 shows a housed state. Esterase derived from leukocytes contained in raw milk was detected using the housing which is housing the laminated filter as a sample testing apparatus and raw milk was the sample.

The product value as raw milk depreciates if the leukocyte count in the raw milk exceeds 300000/ml. Thus, it is necessary to accurately determine the leukocyte count in raw milk. While the difference is 300000 and 400000 in 1 ml, the amount of raw milk contained in a test chip is 0.1 ml. The color intensities for 30000 and 40000 in terms of leukocyte count must be differentiated. In this regard, a test was conducted by changing the number of sheets of non-woven fabric used as a site of reaction as follows.

TABLE 1

| | 2 sheets | 3 sheets | 4 sheets | 5 sheets | 6 sheets |
|---|---|---|---|---|---|
| Identification by | Not | Not | Identi- | Considerably | Very |
| visual inspection | identi- | identi- | fiable | clear | clear |
| | fiable | fiable | | | |

It was found as a result thereof that color intensities due to the difference in the leukocyte count of 30000 and 40000 can be detected by using 4 or more filters, and the difference in the number of leukocytes contained in raw milk can be identified considerably clearly by setting 5 or more filters.

### Example 2: Use of Filters with Different Shapes

The effect of combining different shapes, i.e., quadrangular and circular, as filters used in a laminated filter was then tested. The same conditions as those in Example 1 were used, except for the difference in the shapes of filters. The diameter of the circular filter was configured to have the same size as the observation window. The position of the filter was adjusted to be at the center of the observation window. This was prepared as a sample testing apparatus. FIG. 4 shows a laminated filter being housed. FIG. 5 shows overlap of 7 filters. The following are the results of measuring somatic cells in raw milk.

TABLE 2

| Number of filters | 5 filters | 7 filters |
|---|---|---|
| Identification by | Clearly | Clearly |
| visual inspection | identifiable | identifiable |

As a result, the difference in color development between 300000 and 400000 leukocytes in 1 ml of raw milk was clearly observed.

### Example 3: Study on Filters

The filter size was specified as follows to prepare a sample testing apparatus. Circular filters were configured with a diameter of 8φ and 10φ, and quadrangular filters were configured as a square with a 12 ml side. Two 10φ circular filters, 1 8φ circular filter, and 3 square filters were combined and laminated in the order of square-square-8φ circular-square-12φ circular-12φ circular from the bottom. The top circular filter was impregnated with a substrate of esterase, i.e., tosyl L-alanine ethyl ester. The sheet underneath was left blank. All of the sheets thereunder were placed while being impregnated with 1M Tris-HCl buffer and dried. This was used as a sample testing apparatus and immersed for 5 seconds in raw milk containing the following number of leukocytes. The apparatus was then incubated on a flat surface. The change in color in the observation window after 5 to 6 minutes was compared with a color chart corresponding to leukocyte count to estimate the leukocyte count in the raw milk.

TABLE 3

| Leukocyte count | 60000 | 440000 | 1510000 |
|---|---|---|---|
| Comparison | Good | Clear | Clear |
| with chart | | | |

The results thereof are shown in Table 3.

### Example 4: Verification of Amplification Effect in Enzyme Immunoassay

Since CRP in the blood is generated by an inflammatory reaction in the body during an infection, this is a good method for qualitatively confirming the presence/absence of an infection or the like.

Recently, it was found that cancer progression and the like can be monitored by measuring the value of CRP in blood more precisely to a lower level, which was determined up to this point as negative. However, since a low level of CRP cannot be detected with currently available reagents, development of a more precise reagent is required, but this is beyond the original objective of estimating a symptom by simply monitoring CRP.

In this regard, since the reference value (normal value) of CRP is 0.5 to 1.0 mg/dl, the current framework was used to study whether a concentration less than or equal to the reference value can be detected.

As for the chip structure, a chip was prepared by combining 10φ and 8φ circular filters and a 12 millimeter square filter, immobilizing an antibody to CRP thereon, and affixing the combination to a housing with a hole on the top and bottom. 0.1 ml of a CRP diluent used as an antigen was dropped into the observation window of the chip. After allowing a reaction to take place for 5 minutes, 1 ml of washing buffer was further dropped into the observation window, and B/F was sufficiently separated. The overflowing washing buffer was discharged from a small-pore provided on the opposite side of the observation window. 0.1 ml of peroxidase labeled second antibody was then dropped in and allowed to react for 5 minutes. A color development reagent was then dropped in to check the presence/absence of color development at each CRP concentration. The results are shown in the following table.

TABLE 4

| CRP concentration | 0.05 | 0.1 | 0.2 | 0.5 |
|---|---|---|---|---|
| Detection | − | ± | + | + |

In this manner, the present invention using a plurality of filters enabled detection of CRP at a higher sensitivity.

As disclosed above, the present invention is exemplified by the use of its preferred embodiments. However, it is understood that the scope of the present invention should be interpreted based solely on the Claims. It is also understood that any patent, any patent application, and any other references cited herein should be incorporated herein by reference in the same manner as the contents are specifically described herein.

### INDUSTRIAL APPLICABILITY

The present invention provides a laminated filter, a sample testing apparatus, and a kit for detecting and/or quantifying a substance subjected to testing in a sample (especially in a liquid sample) with sufficient a level of measurement sensitivity and precision as well as convenience.

1. A laminated filter for detecting and/or quantifying a substance subjected to testing, the laminated filter comprising at least four filters, wherein at least two of the at least four filters comprise a reaction reagent that reacts with the substance subjected to testing wherein the at least four filters comprise a first filter and a second filter, the first filter being n-gonal, and the second filter being m-gonal or circular, wherein n is an integer from 3 to 6 and m is greater than n.

2.-4. (canceled)

5. The laminated filter of claim 1, wherein the first filter is adjacent to the second filter.

6. The laminated filter of claim 1, wherein the first filter and the second filter have different areas.

7. The laminated filter of claim 1, wherein the laminated filter comprises a plurality of the first filters and a plurality of the second filters, and at least one of the first filters is adjacent to at least one of the second filters.

8. The laminated filter of claim 5, wherein the first filter and the second filter are alternately laminated.

9. The laminated filter of claim 7, wherein at least two of the plurality of the first filters are laminated after being rotated so that positions of vertices are different.

10. The laminated filter of claim 7, wherein the plurality of the second filters are m-gonal, and at least two of the second filters are laminated after being rotated so that positions of vertices are different.

11. The laminated filter of claim 1, wherein the first filter is quadrangular, and the second filter is circular.

12. The laminated filter of claim 1, wherein the filter is a filter selected from the group consisting of filter paper, non-woven fabric, and glass fiber.

13. The laminated filter of claim 1, wherein a reaction between the substance subjected to testing and the reaction reagent is a color reaction.

14. The laminated filter of claim 1, wherein the substance subjected to testing is an enzyme, and the reaction reagent comprises a substrate of the enzyme.

15. The laminated filter of claim 1, wherein the substance subjected to testing is esterase, and the reaction reagent comprises a substrate of esterase.

16. The laminated filter of claim 15, wherein the reaction reagent comprises 3-[N-(p-toluenesulfonyl)-L-alanyloxy]indole.

17. The laminated filter of claim 1, wherein at least one of the plurality of filters comprises a reaction promoting agent.

18. A sample testing apparatus comprising the laminated filter of claim 1 and a housing for the laminated filter.

19. The sample testing apparatus of claim 18 for measuring a leukocyte in raw milk.

20. The sample testing apparatus of claim 18, wherein the housing comprises a syringe.

21. A kit comprising the sample testing apparatus of claim 18.
